(19) United States
(12) Patent Application Publication
(10) Pub. No.: US 2006/0030550 A1
(43) Pub. Date: Feb. 9, 2006

(54) Pharmaceutical formulations

(76) Inventors: Theodore L. Lithgow, Bridgewater, NJ (US); Paul T. Medeiros, Easton, PA (US); Keith Anthony Ellway, Kinnelon, NJ (US); Thomas J. Higgins, Chatham, NJ (US); Richard R. Lorber, Scotch Plains, NJ (US); Bruce A. Malcolm, Westfield, NJ (US); Elaine Radwanski, Hasbrouck Hts., NJ (US); Heribert W. Staudinger, Union, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/196,745

(22) Filed: Aug. 3, 2005

Related U.S. Application Data
(60) Provisional application No. 60/598,618, filed on Aug. 4, 2004.

(51) Int. Cl. A61K 31/573; A61K 31/4245

(52) U.S. Cl. 514/171; 514/364

(57) ABSTRACT

Disclosed are medicaments containing, separately or together, (A) Pleconaril or a pharmaceutically acceptable salt thereof and (B) a pharmaceutically active agent for simultaneous, sequential or separate administration in the treatment of a viral infection and/or other disease states and the symptoms associated therewith.

### CROSS REFERENCE TO RELATED APPLICATION

This application claims benefit of priority to U.S. Provisional Patent Application Ser. No. 60/598,618, the entirety of which is incorporated by reference.

### PHARMACEUTICAL FORMULATIONS

The present invention is directed to formulations containing Pleconaril either alone or in combination with one or more other pharmaceutically active ingredients in novel dosage forms and methods of using the same.

### BACKGROUND OF THE INVENTION

Pleconaril is known as 1,2,4-oxadiazole3-[3,5-Dimethyl-4-[3-(3-methyl-5-isoxazolyl)propoxy]phenyl]-5-(trifluoremethyl). It has other names such as PICOVIR®, VP 63843 and Win 63843. It has been shown to be active against rhinoviruses. Due to the efficacy of Pleconaril as an anti-viral agent for the treatment of the common cold, it would be beneficial to administer it along with other medications and/or in certain dosage forms that relieve symptoms associated with the common cold, viral induced respiratory diseases and/or other disease states.

### SUMMARY OF THE INVENTION

Accordingly, there is disclosed medicament containing, separately or together, (A) Pleconaril or a pharmaceutically acceptable salt thereof and (B) a corticosteroid, for simultaneous, sequential or separate administration in the treatment of an upper or lower respiratory, viral, inflammatory or obstructive airways disease. Preferably, the corticosteroid is Mometasone Furoate monohydrate.

There is also disclosed a medicament containing, separately or together, (A) Pleconaril or a pharmaceutically acceptable salt thereof and (B) an antihistamine, for simultaneous, sequential or separate administration in the treatment of an upper or lower respiratory, viral, inflammatory or obstructive airways disease. Preferably, the antihistamine is Desloratadine or loratadine.

There is also disclosed a medicament containing, separately or together, (A) Pleconaril or a pharmaceutically acceptable salt thereof and (B) an expectorant, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) an NSAID, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) a decongestant for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) an anti-cholinergic, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) zinc, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) an antibiotic, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) an H₃ antagonist, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) a Leukotriene₄ antagonist, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) an P2Y₂ agonist, for simultaneous, sequential or separate administration in the treatment of an upper or lower respiratory, viral, inflammatory or obstructive airways disease.

There is also disclosed a medicament containing, separately or together, (A) Pleconaril or a pharmaceutically acceptable salt thereof and (B) a syk kinase antagonist, for sequential or separate administration in the treatment of an upper or lower respiratory, viral, inflammatory or obstructive airways disease.

It is believed that certain of the medicaments of the present invention will have advantages over medicaments containing only pleconaril as the active agent. For example, it is believed that the medicaments of the present invention containing Pleconaril and one or more active agents described above when formulated together for administration using a nebulizer have advantages including but not limited to oral administration, ease of pediatric therapy and/or high dose loading availability in another example, it is believed that the medicaments of the present invention containing Pleconaril and one or more of the other active agents described above can be formulated as a metered dose inhaler product, that may be administered either orally or nasally simply by switching the actuator that is designed ofr nasal delivery with an actuator designed for oral delivery.

### DETAILED DESCRIPTION OF THE INVENTION

Pleconaril is known as 1,2,4-oxadiazole3-[3,5-Dimethyl-4-[3-(3-methyl-5-isoxazolyl)propoxy]phenyl]-5-(trifluoromethyl). It has other names such as PICOVIR®, VP 63843 and Win 63843. Pleconaril is a picornavirus replication inhibitor useful in the treatment of, amongst other things, viral induced infections of the upper and lower airways, viral meningitis, and life-threatening diseases such as chronic meningoencephalitis, neonatal enteroviral disease, polio and myocarditis. According to the Merck Index, it may be prepared in accordance with U.S. Pat. No. 5,464,848, which is incorporated by reference. When used in the medicaments of the present invention, it may be administered in an amount ranging from about 1 mg to about 600 mg, preferably about 200 to about 400 mg in single or divided doses daily for a period sufficient to treat a viral infection, or more particularly, a viral induced respiratory infection.

In one aspect of the present invention, Pleconaril may be combined with a corticosteroid. Corticosteroids for use in the present invention, without limitation, include mometasone furoate, dexamethasone, butoxicort, rofleponide, budesonide, deflazacort, ciclesonide, fluticasone, beclomethasone, loteprednol or triamcinolone.

A particularly preferred corticosteroid is Mometasone Furoate. Mometasone Furoate is a corticosteroid approved for topical dermatologic use to treat inflammatory and/or pruritic manifestations of corticosteroid-responsive dermatoses. The compound may be prepared in accordance with the procedures disclosed in U.S. Pat. Nos. 4,472,393, 4,731,447, 4,873,335, 5,837,699 and 6,127,353, all of which are hereby incorporated by reference in their entirety. Mometasone Furoate is a topically active steroid which is not readily bioavailable. It is commercially available as a spray for intra-nasal administration under the name of Nasonex®. Mometasone's use for the treatment of airway passages and lung diseases is disclosed in U.S. Pat. Nos. 6,677,323, 6,677,322, 6,365,581, 6,187,765, 6,068,832, 6,057,307 5,889,015 5,837,699 and 5,474,759, all of which are incorporated by reference in their entirety.

For the treatment of allergic, non-allergic rhinitis and/or inflammatory diseases of the upper or lower airway passages to treat for example asthma or allergic or non-allergic rhinitis, the substantially non-systematically bioavailable amount of Mometasone Furoate which may be administered as an aqueous suspension or dry powder is in the range of about 10 to 5000 micrograms (“mcg”)/day, 10 to 4000 mcg/day, 10 to 2000 mcg/day, 25-1000 mcg/day, 25 to 400 mcg/day, 25-200 mcg/day, 25-100 mcg/day or 25-50 mcg/day in single or divided doses.

For instance, when the corticosteroid is fluticasone, it may be administered at the dose of 2 sprays of 50 μg of fluticasone propionate each in each nostril once daily. Alternatively, it may be administered at a dose of fluticasone is 1 spray of 50 μg of fluticasone propionate each in each nostril once daily. When the corticosteroid is triamcinolone, it may be administered at a dose of triamcinolone is 220 μg per day as two sprays in each nostril once daily. Alternatively, it may be administered at a dose of 110 μg per day as one spray in each nostril once daily. When the corticosteroid is budesonide, the administered dose of budesonide may be 64 μg per day administered as one spray per nostril of 32 μg once daily.

In another aspect of the present invention, Pleconaril can be combined with an histamine H₁ receptor antagonist that is selected from the group consisting of Astemizole, Azatadine, Azelastine, Acrivastine, Bromphemiramine, Chlorpheniramine, Clemastine, Cyclizine, Carebastine, Cyproheptadine, Carbinoxamine, Desloratadine, Doxylamine, Diphenhydramine, Cetirizine, Dimenhydrinate, Dimethindene, Ebastine, Epinastine, Efletirizine, Fexofenadine, Hydroxyzine, Ketotifen, Loratadine, Levocabastine, Levocetirizine, Mizolastine, Mequitazine, Mianserine, Noberastine, Meclizine, Norastemizole, Picumast, Pyrilamine, Promethazine, Terfenadine, Tripelennamine, Temelastine, Trimeprazine, Triprolidine and mixtures of any two or more of the foregoing. The Pleconaril and antihistamine(s) may be administered both orally and topically. Topical and oral administration may be carried out as set forth herein.

One preferred antihistamine to be combined with Pleconaril is Desloratadine, or Descarboethoxyloratidine or DCL. DCL is a non-sedating antihistamine, whose technical name is 8-chloro-6,11-dihydro-11-(4-piperidylidene)-5H-benzo[5,6]cyclohepta[1,2]pyridine. This compound is described in Quercia, et al., Hosp. Formul., 28: 137-53 (1993), in U.S. Pat. No. 4,659,716, and in WO 96/20708. The use of Desloratadine for the treatment of congestion is disclosed in U.S. Pat. No. 6,432,972. DCL is an antagonist of the H₁ histamine receptor protein. The H₁ receptors are those that mediate the response antagonized by conventional antihistamines. H₁ receptors are present, for example, in the ileum, the skin, and the bronchial smooth muscle of man and other mammals. The amount of DCL which can be employed in a unit (i.e. single) dosage form of the present compositions can range from about 2.5 to about 45 mg, also from about 2.5 to about 20 mg, also from about 5 to about 10 mg. Preferred dosage amounts include 2.5 mg, 5.0 mg, 10.0 mg and 20.0 mg.

Another antihistamine for use with Pleconaril is Loratadine. Loratadine is a non-sedating antihistamine whose technical name is 11-(4-piperidylidene)-5H-benzo-[5,6]-cyclohepta-[1,2-b]-pyridine. The compound is described in U.S. Pat. No. 4,282,233. Loratadine is a potent tricyclic and antihistaminic drug of slow release, with a selective antagonist of peripheric H₁ receptors activity.

Another antihistamine for use with Pleconaril is Fexofenadine. Fexofenadine reportedly is a non-sedating antihistamine, whose technical name is 4-[1-hydroxy-4-(4-hydroxy-diphenylmethyl)-1-piperidinyl)butyl]-α, α-dimethyl-benzene acetic acid. Preferably the pharmaceutically acceptable salt is the hydrochloride, also known as fexofenadine hydrochloride. The amount of fexofenadine which can be employed in a unit dosage form of the present composition can range from about 40 to 200 mg, also from about 60 to about 180 milligrams, also about 120 milligrams.

Another antihistamine for use with Pleconaril is Cetirizine. Cetirizine hydrochloride reportedly is an H₁ receptor antagonist. The chemical name is (±)-[2-[4-[(4-chlorophenyl)phenylmethyl]-1-piperazinyl] ethoxy]acetic acid, dihydrochloride. Cetirizine hydrochloride is a racemic compound with an empirical formula of C₂₁H₂₅ClN₂O₃.2HCl. Cetirizine hydrochloride is a white, crystalline powder and is water soluble. Cetirizine hydrochloride is available from Pfizer Inc., New York, N.Y., under the trade name ZYRTEC®. The amount of Cetirizine which can be employed in a unit dosage form of the present composition can range from about 0 to 40 mg, also from about 5 to about 10 milligrams. The levo isomer of Cetirizine may also be combined with Pleconaril in the formulations of the present invention. Another form of Cetirizine for use in the present invention is Cetirizine dinitrate.

Also for use within the scope of the present invention is the use of expectorants in combination with Pleconaril. Ambroxol is a bromhexine metabolite, chemically identified as trans-4(2-amino-3,5-dibromobenzil, amine) ciclohexane hydrochloride, which has been widely used during more than two decades as an expectorant agent or stimulating pulmonary surfactant factor. The compound is described in U.S. Pat. No. 3,536,712. Guaiafenesin is an expectorant, whose technical name is 3-(2-methoxyphenoxy)-1,2-propanediol. The compound is described in U.S. Pat. No. 4,390,732. Terpin hydrate is an expectorant, whose technical name is 4-hydroxy-α, α, 4-trimethylcyclohexane-methanol. Potassium guaicolsulfonate is an expectorant, whose technical name is 3-Hydroxy-4-methoxybenzenesulfonic acid mix with mono-potassium 4-hydroxy-3-methoxybenzenesulfonate. These combinations with Pleconaril may be administered orally as set forth below.

Also for use within the scope of the present are both oral and nasal decongestants in combination with Pleconaril. The nasal decongestants useful in the present invention include the sympathomimetic amine nasal decongestants. Those currently approved for topical use in the United States include, without limitation, levmetamfetamine (also known as 1-desoxyephedrine), ephedrine, ephedrine hydrochloride, ephedrine sulfate, naphazoline hydrochloride, oxymetazoline hydrochloride, phenylephrine hydrochloride, propylhexedrine and xylometazoline hydrochloride. Oral decongestants for use in the present invention include phenylpropanolamine, phenylephrine and pseudoephedrine. Pseudoephedrine as well as pharmaceutically acceptable acid additional salts, e.g., those of HCl or H₂SO₄, is a sympathomimetic drug recognized by those skilled in the art as a safe therapeutic agent effective for treating nasal congestion and is commonly administered orally and concomitantly with an antihistamine for treatment of nasal congestion associated with allergic rhinitis. The use of pseudoephedrine as a nasal decongestant in the present invention is preferred in amounts of about 120 mg pseudoephedrine sulfate dosed one to 4 times daily. However, lesser amounts of pseudoephedrine sulfate may be used in combination with Pleconaril.

Specific drugs in combination with Pleconaril that may be incorporated when the composition is intended to relieve oropharyngeal discomfort, such as sore throat, cold or canker sores, painful gums and other conditions are topical anesthetics such as phenol, hexylresorcinol, salicyl alcohol, benzyl alcohol, dyclonine, dibucaine, benzocaine, buticaine, cetylpyridinium chloride, diperidon, clove oil, menthol, camphor, eugenol and others. Similarly, drugs that may be incorporated for application to the skin for relieving discomfort include lidocaine, benzocaine, tetracaine, dibucaine, pramoxine, diphenhydramine, benzyl alcohol and others.

Also for use in combination with Pleconaril are histamine H₃ receptor antagonists. Currently known histamine H₃ receptor include, without limitation: Thioperamide, Impromidine, Burimamide, Clobenpropit, Impentamine, Mifetidine, S-sopromidine, R-sopromidine, 3-(imidazol-4-yl)-propylguanidine (SKF-91486), 3-)(4-chlorophenyl)methyl-5->2-(1H-imidazol-4yl)ethyl 1,2,3-oxadiazole (GR-175737), 4-(1-cyclohexylpentanoyl-4-piperidyl) 1H-imidazole (GT-2016), 2-{>2->4(5)-imidazolylethylthio}-5-nitropyridine (UCL-1199) Clozapine, SCH497079 and SCH539858. Particularly preferred compounds are disclosed and claimed in U.S. Pat. No. 6,720,328 and United States Patent Application Publication No. 20040097483A1, both assigned to Schering Corp., and both of which are hereby incorporated by reference. Other preferred compositions may further include both H₁ and H₃ receptors antagonists as is disclosed in U.S. Pat. No. 5,869,479, also assigned to Schering Corp., which is hereby incorporated by reference. Other compounds can readily be evaluated to determine activity at H₃ receptors by known methods, including the guinea pig brain membrane assay and the guinea pig neuronal ileum contraction assay, both of which are described in U.S. Pat. No. 5,352,707. Another useful assay utilizes rat brain membranes and is described by West et al., “Identification of Two H₃-Histamine Receptor Subtypes,” Molecular Pharmacology, Vol. 38, pages 610-613 (1990).

Other agents for use with Pleconaril within the scope of the present invention include Anti-Cholinergic agents. Particularly preferred agents include Tiotropium, Oxitropium, Ipratropium, Methantheline, Propantheline, Dicyclomine, Scopolamine, Methscopolamine, Telenzepine, Benztropine, QNX-hemioxalate, Hexahydro-sila-difenidol hydrochloride and Pirenzepine. These compositions may be administered either orally or nasally as set forth below in amounts that are known to one of skill in the art.

Antibiotics for use in combination with Pleconaril in the present invention include antibiotics such as Tetracycline, Chlortetracycline, Bacitracin, Neomycin, Polymyxin, Gramicidin, Oxytetracycline, Chloramphenicol, Florfenicol, Gentamycin, Erythromycin, Clarithromycin, Azithromycin, Tulathromycin, or other suitable macrolide, Cefuroxime, Ceftibuten, Ceftiofur, Cefadroxil, or other suitable cephalosporin, Amoxicillin, Peniccilins, Amoxicillin with clavulanic acid or an other suitable beta-lactamase inhibitor, antibacterials such as Sulfonamides, Sulfacetamide, Sulfamethizole, Sulfisoxazole; Nitrofurazone, and Sodium propionate. These compositions may be administered either orally or nasally as set forth below in amounts that are known to one of skill in the art.

Other agents for use within the scope of the present invention include P2Y₂ receptor agonists in combination with Pleconaril. Diquafosol tetrasodium is a P2Y₂ receptor agonist that activates receptors on the ocular surface and inner lining of the eyelid to stimulate the release of water, salt, mucin and lipids—the key components of natural tears. Mucin is made in specialized cells and acts to lubricate surfaces. Lipids in the eye are oily substances that form the outer-most layer of the tear film and are responsible for the prevention of excess tear fluid evaporation. In preclinical testing, diquafosol reportedly increased the secretions of natural tear components. Diquafosol is available from Inspire. P2Y₂ receptor agonists are a new class of compounds that are being developed for the treatment of a variety of conditions in which MCC is impaired, including chronic bronchitis and CF. Other mucolytic agents may include N-Acetylcysteine and endogenous ligand compound UTP. These compositions may be administered either orally or nasally as set forth below in amounts that are known to one of skill in the art.

Also for use with Pleconaril in the present invention are Non-Steroidal Anti-Inflammatory (“NSAID's”) agents. Suitable NSAID's include Acetylsalicylic acid, Acetaminophen, Indomethacin, Diclofenac, Piroxicam, Tenoxicam, Ibuprofen, Naproxen, Ketoprofen, Nabumetone, Ketorolac, Azapropazone, Mefenamic acid, Tolfenamic acid, Sulindac, Diflunisal, Tiaprofenic acid, Podophyllotoxin derivatives, Acemetacin, Aceclofenac, Droxicam, Oxaprozin, Floctafenine, Phenylbutazone, Proglumetacin, Flurbiprofen, Tolmetin and Fenbufen. These compositions may be administered either orally or nasally as set forth below in amounts that are known to one of skill in the art.

Also for use with Pleconaril in the present invention are Leukotriene₄ Antagonists. Suitable leukotriene D₄ antagonists included Zileuton, Docebenone, Piripost, ICI-D2318, MK-591, MK-886, sodium 1-(((R)-(3-(2-(6,7-difluoro-2-quinolinyl)ethynyl)phenyl)-3-(2-(2-hydroxy-2-propyl)phenyl)thio)methyl)cyclopropane-acetate; 1-(((1 (R)-(3-(2-(2,3-dichlorothieno[3,2-b]pyridin-5-yl)-(E)-ethenyl)phenyl)-3-(2-(1-hydroxy-1-methylethyl)phenyl)propyl)thio)-methyl)cyclopropaneacetic acid, Pranlukast, Zafirlukast, and Montelukast. These compositions may be administered either orally or nasally as set forth below in amounts that are known to one of skill in the art.

Montelukast is a Leukotriene D₄ antagonist capable of antagonizing the receptors for the cysteinyl leukotrienes. The technical name of Montelukast is [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid. This compound is described in EP 480,717. A preferred pharmaceutically acceptable salt of Montelukast is the monosodium salt, also known as Montelukast sodium. The amount of Montelukast which can be employed in a unit dosage form of the present invention can range from about one to 100 milligrams, also from about 5 to about 20 milligrams, preferably about 10 milligrams.

The compound 1-(((R)-(3-(2-(6,7-difluoro-2-quinolinyl)ethenyl)phenyl)-3-(2-(2-hydroxy-2-propyl)phenyl)thio)methylcyclopropaneacetic acid is a leukotriene antagonist described in WO 97/28797 and U.S. Pat. No. 5,270,324. A pharmaceutically acceptable salt of this compound is the sodium salt, also known as sodium 1-(((R)-(3-(2-(6,7-difluoro-2-quinolinyl)ethenyl)phenyl)-3-(2-(2-hydroxy-2-propyl) phenyl)thio)-methylcyclopropaneacetate.

The compound 1-(((1 (R)-3-(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetic acid is a leukotriene antagonist described in WO 97/28797 and U.S. Pat. No. 5,472,964. A pharmaceutically acceptable salt of this compound is the sodium salt, also known as sodium 1-(((1 (R)-3-(3-(2-(2,3-dichlorothieno[3,2-b]pyridin-5-yl)-(E)-ethenyl)phenyl)-3-(2-(1-hydroxy-1-methylethyl)phenyl)propyl)-thio)methyl)cyclopropaneacetate.

Praniukast is a leukotriene antagonist described in WO 97/28797 and EP 173,516. The technical name for this compound is N-[4-oxo-2-(1H-tetrazol-5-yl)-4H-1-benzopyran-8-yl]-p-(4-phenylbutoxy)benzamide. The amount of Pranlukast which can be employed in a unit dosage form can range from about 100 to about 700 mg, preferably from about 112 to about 675 mg; also from about 225 mg to about 450 mg; also from about 225 to about 300 mg.

Zafirlukast is a leukotriene antagonist described in WO 97/28797 and EP 199,543. The technical name for this compound is cyclopentyl-3-[2-methoxy-4-[(o-tolylsulfonyl)carbamoyl]benzyl]-1-methylindole-5-carbamate.

The compound [2-[[2-(4-tert-butyl-2-thiazolyl)-5-benzofuranyl]oxymethyl]phenyl]acetic acid is a leukotriene antagonist and/or inhibitor whose method for preparation is described in U.S. Pat. No. 5,296,495 and Japanese Patent JP 08325265 A. An alternative name for this compound is 2-[[[2-[4-(1,1-dimethylethyl)-2-thiazolyl]-5-benzofuranyl]oxy]methyl]-benzeneacetic acid. The code number for this compound is FK011 or FR150011.

Also for use with Pleconaril in the present invention are formulations containing Zinc in solution. Zinc reportedly has beneficial effects against the common cold. The solution comprises a concentration of ionic zinc below that which causes irritation to mucus membranes. The majority of the ionic zinc in the spray is unchelated zinc and is in the form of free ionic solution. The solution contains substantially unchelated zinc ions in a concentration of from about 0.004 to about 0.12% (w/vol), to the nostrils and respiratory tract of a patient in need thereof. The solution can be selected from the group consisting of aqueous and saline solutions; The substantially unchelated ionic zinc compound can comprise a mineral acid salt of zinc; can comprise a salt selected from the group consisting of zinc sulfate and zinc chloride, such as zinc acetate, are generally preferable. These compositions may be administered either orally or nasally as set forth below in amounts that are known to one of skill in the art.

Also for use with Pleconaril in the present invention are compounds known for the treatment of the common cold such as echinacea, Vitamin C, Vitamin E, anti-oxidants and the like.

Also for use with Pleconaril in the present invention are a class of molecules which work via a novel mechanism, blocking syk kinase, such as R112, available from Rigel Pharmaceuticals, Inc. A recent study reportedly showed a greater than 20% relative improvement for R112 over placebo (an absolute difference of 9% over placebo) and up to 38% improvement for R112 from baseline measurements (prior to drug initiation). In particular, symptoms most closely associated with chronic nasal congestion (e.g. stuffy nose) were reportedly improved with R112 over placebo.

Also for use with Pleconaril in the present invention are 5-lipoxygenase inhibitors. The term “5-lipoxygenase inhibitor” or “5-LO inhibitor” includes any agent, or compound that inhibits, restrains, retards or otherwise interacts with the enzymatic action of 5-lipoxygenase, such as, but not limited to, zileuton, docebenone, piripost, and the like. The term “5-lipoxygenase activating protein antagonist” or “FLAP antagonist” includes any agent or compound that inhibits, retrains, retards or otherwise interacts with the action or activity of 5-lipoxygenase activating protein, 10 such as, but not limited to MK-591 and MK-886.

As will be evident to one of skill in the art, the formulations of the present invention may contain Pleconaril either alone or in combination with one or more pharmacologically active agents as set forth herein. For instance, the formulation may contain Pleconaril in combination with Desloratadine and/or Pseudoephedrine for the treatment of a respiratory, viral, inflammatory or obstructive airways disease.

The devices found useful for providing measured substantially non-systematically bioavailable amounts of aerosolized pharmaceutical compositions thereof for delivery to the oral airway passages and lungs by oral inhalation or intranasally by inhalation include pressurized metered-dose inhalers (“MDI”) which deliver aerosolized particles suspended in chlorofluorocarbon propellants such as CFC-11, CFC-12, or the non-chlorofluorocarbons or alternate propellants such as the fluorocarbons, HFC-134A or HFC-227 with or without surfactants.

In another embodiment, the formulations of the present invention may be also administered in specific, measured amounts in the form of an aqueous suspension by use of a pump spray bottle such as the bottles used to deliver NASONEX® Nasal Spray as well as the spray bottle disclosed in the Schering Corporation Industrial Design Deposit DM/026304, registered by the Hague Union on Jun. 1, 1993 (each are available from Schering Corporation).

In another embodiment of the present invention, the delivery device may comprise 2 interchangeable actuators, respectively, for both oral and nasal delivery to treat both the oral and nasal sites of viral activity. A typical actuator for nasal delivery may be circular with an orifice diameter of about one millimeter. An actuator for use in oral delivery can be enclosed within a mouthpiece and the actuator typically has an orifice diameter of about 0.5 millimeters.

The formulations of the present invention may also be administered via a Dry Powder Inhaler. Such inhalers include, without limitation, Schering's Twisthaler, Diskhaler (Allen & Hanburys), Accuhaler (Allen & Hanburys), Diskus (Glaxo), Spiros (Dura), Easyhaler (Orion), Cyclohaler (Pharmachemie), Cyclovent (Pharmachemie), Rotahaler (Glaxo), Spinhaler (Fisons), FlowCaps(Hovione), Turbospin (PH&T), Turbohaler (Astra), EZ Breath (Norton Healthcare/IVAX), MIAT-HALER (Miat), Pulvinal (Chiesi), Ultrahaler (Fisons/Rhone Poulenc Rorer), MAG-Haler (GGU), Prohaler (Valois), Taifun (Leiras), JAGO DPI (JAGO), and M L Laboratories' DPI (M L Laboratories).

The formulations of the present invention may also be administered via a nebulizer device. Typical commercial nebulizer devices produce dispersions of droplets in gas streams by one of two methods. Jet nebulizers use a compressed air supply to draw up a fluid by venturi action and introduce it into a flowing gas stream, after which the fluid is caused to impact one or more stationary baffles to remove excessively large droplets. Ultrasonic nebulizers use an electrically driven transducer to subject a fluid to high-frequency oscillations, producing a cloud of droplets which can be entrained in a moving gas stream; these devices are less preferred for delivering suspensions. There are hand-held nebulizers which atomize the fluid with a squeeze bulb air supply, but the more widely used equipment incorporates an electrically powered compressor or connects to a cylinder of compressed gas. Although the various devices which are commercially available vary considerably in their delivery efficiency for a given medicament, they all are useful for the treatment of the present invention; it is necessary for the prescriber to specify an exact amount of medicament formulation which is to be charged to each particular device, since their respective outputs of respirable droplets are far from identical.

Suspension formulations suitable for nebulization must, of course, contain solid particles of a respirable size (e.g., preferably averaging less than about 5 .mu.m in the largest dimension and more preferably averaging less than about 2 .mu.m) and must maintain their suspended particle size distribution during storage. In addition, the particle-containing droplets formed during nebulization of the formulations must have appropriate sizes for deposition in the desired area of the respiratory system.

As is known to one of skill in the art, the product may contain 2 interchangeable actuators. For instance, it could contain one actuator for nasal administration and one actuator for oral administration.

For oral dosage form preparations, a pharmaceutically acceptable carrier (which includes diluents, excipients or carrier materials) is also present in the composition. The carrier is suitably selected with respect to the intended form of administration, i.e. oral tablets, capsules (either solid-filled, semi-solid filled or liquid filled), powders for constitution, oral gels, elixirs, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, mannitol, ethyl alcohol (liquid forms) and the like. Moreover, when desired or needed, suitable binders, lubricants, disintegrating agents, disinfectants and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like. Disinfectants include benzalkonium chloride and the like. Sweetening and flavoring agents and preservatives may also be included where appropriate.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Dosage form—refers to composition containing the antihistamine and the carrier formulated into a delivery system, i.e., tablet, capsule, oral gel, powder for constitution or suspension in association with inactive ingredients.

Capsule—refers to a special container or enclosure made of methyl cellulose, polyvinyl alcohols, or denatured gelatins or starch for holding or containing compositions comprising the antihistamine and the carrier. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the ingredients (the antihistamine and the carrier) with suitable diluents. The tablet can be prepared by compression of mixtures or granulations obtained by wet granulation, dry granulation or by compaction.

Oral gels-refers to the antihistamine and the carrier dispersed or solubilized in a hydrophilic semi-solid matrix.

Powders for constitution refers to powder blends containing the antihistamine and the carrier and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidinone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d'l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidants—materials that prevent caking and improve the flow characteristics of granulations, so that flow is smooth and uniform. Suitable glidants include silicon dioxide and talc. The amount of glidant in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control, as well as to topical bioavailability.

Conventional methods for preparing tablets are known. Such methods include dry methods such as direct compression and compression of granulation produced by compaction, or wet methods or other special procedures.

For opthalmic compositions, the compositions of the present invention may take various forms. For example, they may be an aqueous gel or liquid, or an ointment. In a preferred embodiment, the composition is a water-in-oil emulsion with the active ingredients in the aqueous droplets suspended in a lotion or flowable ointment base comprising, e.g., petrolatum, mineral oil, and the like. Additional emollient ingredients such as isopropyl myristate may also be added. Such a lotion or ointment covers the conjunctiva and cornea with a thin film that both carries active ingredients and provides for prolonged drainage through the naso-lacrimal ducts. The film also provides a barrier to evaporative loss of water from the corneal stroma.

The term “pharmaceutically acceptable salt” refers to a salt prepared from pharmaceutically acceptable non-toxic acids or bases including inorganic acids or bases or organic acids or bases. Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine and procaine.

The Pleconaril and active agent combination and the optional ingredient or ingredients may be administered in combination or separately in the method of treating the disease. For example, they may be administered concurrently or sequentially, i.e. they may be administered in combination either concurrently or by the sequential administration of the ingredients in a suitable order.

The phrase “therapeutically effective amount” means that amount of the Pleconaril and one or more pharmaceutically active agents which provides a therapeutical benefit in the treatment or management of the disease or disease states.

The compositions of the present invention may be used for the treatment of a number of viral based disorders including the treatment and/or prevention of the common cold. The compositions of the present invention may be used for the prevention of exacerbation of disorders of the upper and lower airways such as allergic rhinitis, congestion associated therewith nasal blockage associated therewith, asthma, chronic obstructive pulmonary disorder, allergic asthma, emphysema, to prevent the need for rescue the use of rescue medications for disorders of the lower airways, sinusitis, fungal induced sinusitis, bacterial based sinusitis, polyposis and the like. The formulations of the present invention may also be used for viral based post exposure treatment.

The compositions may also be used prophylactically when a household member, typically a child, is stricken with a cold. Alternatively, it may be used in settings where there is a high incidence of viral or bacterial based pathogens such as in a hospital, nursing home, pharmacy and the like. The compositions of the present invention may also be used prophylactically to prevent exacerbations of symptoms associated with diseases of the upper airways in individuals with such diseases.

For disorders of the lower airways, the severity of the disease state in a patient can be quantified by objective pulmonary function tests, including a measurement of the patient's forced expiratory volume in 1 second (FEV₁). When this result is about 65 to 79 percent of the predicted value (determined using a formula that takes into account the patient's age and size), the airway obstruction is considered to be mild. For an FEV₁ value about 50 to 64 percent of predicted, the airway obstruction is classified as moderate; if the value is less than 50 percent of predicted, the airway obstruction is considered to be severe; and if the value is less than 30 percent the airway obstruction is considered to be very severe. This test utilizes relatively simple and inexpensive equipment, and therefore is widely used for disease state diagnosis, and to monitor the progression lung and airway disorders during treatment.

For diseases of the upper airways, there are also objective parameters for measuring an improvement in symptoms. Efficacy endpoints studies may include were Total Symptom Score, Total Nasal Symptom Score, Total Non-nasal Symptom Score, and Health Quality of Life (HQOL) analysis in efficacy trials. The compositions of the present invention may be tested for reducing the total symptom scores (the sum of individual scores for rhinorrhea, sneezing, congestion/stuffiness, nasal itching, itchy/burning eyes, tearing, ocular redness, and itchy ears/palate). An important efficacy endpoint that may be analyzed in the studies is the AM NOW total symptom score. This parameter measures the total symptom relief by the patient after 24 hours before taking the next day dose.

The compositions of the present invention may be particularly useful for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal and perennial allergic rhinitis, including nasal congestion, in patients in need of such treating and/or preventing.

The foregoing descriptions of various embodiments of the invention are representative of various aspects of the invention, and are not intended to be exhaustive or limiting to the precise forms disclosed. Many modifications and variations undoubtedly will occur to those having skill in the art. It is intended that the scope of the invention shall be fully defined solely by the appended claims.

We claim:

1. A medicament containing, separately or together, (A) Pleconaril or a pharmaceutically acceptable salt thereof and (B) a corticosteroid, for simultaneous, sequential or separate administration in the treatment of an upper or lower respiratory, viral, inflammatory or obstructive airways disease.

2. A medicament according to claim 1, which is a pharmaceutical composition comprising a mixture of effective amounts of (A) and (B), optionally together with a pharmaceutically acceptable carrier.

3. A medicament according to claim 2, which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

4. A medicament according to claim 3, in which (A) or (B), or (A) and (B), are in dispersion in the propellant, which is a halogen-substituted hydrocarbon.

5. A medicament according to claim 4, in which (A) or (B), or each of (A) and (B), has an average particle diameter of up to 10 μm.

6. A medicament according to claim 3, which is an inhalable nebulizable composition comprising a dispersion of (A) and (B) in an aqueous, organic or aqueous/organic medium or a combination of a dispersion of (A) in said medium with a dispersion of (B) in said medium.

7. A medicament according to claim 2, which is an inhalable dry powder comprising finely divided (A) or (B), or finely divided (A) and (B), optionally together with a pharmaceutically acceptable carrier in finely divided form.

8. A medicament according to claim 7, in which the carrier is present and is a saccharide.

9. A medicament according to claim 8, in which the carrier is lactose.

10. A medicament according to claim 9, in which (A) or (B), or each of (A) and (B), has an average particle diameter up to 10 microns.

11. A pharmaceutical kit comprising (A) as defined in claim 1 and (B) as defined in claim 1 in separate unit dosage forms, said forms being suitable for administration of (A) and (B) in effective amounts, together with one or more inhalation devices for administration of (A) and (B).

12. A medicament according to claim 1, wherein the corticosteroid is one or more selected from the group consisting of Mometasone Furoate, Dexamethasone, Butoxicart, Rofleponide, Budesonide, Deflazacort, Ciclesonide, Fluticasone, Beclomethasone, Loteprednol and Triamcinolone.

13. A medicament according to claim 12, wherein the corticosteroid is Mometasone Furoate.

14. A medicament according to claim 13, wherein the Mometasone Furoate is in an aqueous suspension.

15. A medicament containing, separately or together, (A) Pleconaril or a pharmaceutically acceptable salt thereof and (B) an antihistamine, for simultaneous, sequential or separate administration in the treatment of an upper or lower respiratory, viral, inflammatory or obstructive airways disease.

16. A medicament according to claim 15 which is a pharmaceutical composition comprising a mixture of effective amounts of (A) and (B), optionally together with a pharmaceutically acceptable carrier.

17. A medicament according to claim 15, which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

18. A medicament according to claim 17, in which (A) or (B), or (A) and (B), are in dispersion in the propellant, which is a halogen-substituted hydrocarbon.

19. A medicament according to claim 18, in which (A) or (B), or each of (A) and (B), has an average particle diameter of up to 10 μm.

20. A medicament according to claim 15, which is an inhalable nebulizable composition comprising a dispersion of (A) and (B) in an aqueous, organic or aqueous/organic medium or a combination of a dispersion of (A) in said medium with a dispersion of (B) in said medium.

21. A medicament according to claim 15, which is an inhalable dry powder comprising finely divided (A) or (B), or finely divided (A) and (B), optionally together with a pharmaceutically acceptable carrier in finely divided form.

22. A medicament according to claim 21, in which the carrier is present and is a saccharide.

23. A medicament according to claim 22, in which the carrier is lactose.

24. A medicament according to claim 21, in which (A) or (B), or each of (A) and (B), has an average particle diameter up to 10 microns.

25. A pharmaceutical kit comprising (A) as defined in claim 15 and (B) as defined in claim 15 in separate unit dosage forms, said forms being suitable for administration of (A) and (B) in effective amounts, together with one or more inhalation devices for administration of (A) and (B).

26. A medicament according to claim 15, wherein the antihistamine is one or more selected from the group consisting of Astemizole, Azatadine, Azelastine, Acrivastine, Bromphemiramine, Chlorpheniramine, Clemastine, Cyclizine, Carebastine, Cyproheptadine, Carbinoxamine, Desloratadine, Doxylamine, Diphenhydramine, Cetirizine, Cetirizine dinitrate, Dimenhydrinate, Dimethindene, Ebastine, Epinastine, Efletirizine, Fexofenadine, Hydroxyzine, Ketotifen, Loratadine, Levocabastine, Levocetirizine, Mizolastine, Mequitazine, Mianserine, Noberastine, Meclizine, Norastemizole, Picumast, Pyrilamine, Promethazine, Terfenadine, Tripelennamine, Temelastine, Trimeprazine, Triprolidine and mixtures of any two or more of the foregoing.

27. A medicament according to claim 26, wherein the antihistamine is Desloratadine.

28. A medicament containing, separately or together, (A) Pleconaril or a pharmaceutically acceptable salt thereof and (B) an expectorant, for simultaneous, sequential or separate administration in the treatment of an upper or lower respiratory, viral, inflammatory or obstructive airways disease.

29. A medicament according to claim 28 which is a pharmaceutical composition comprising a mixture of effective amounts of (A) and (B), optionally together with a pharmaceutically acceptable carrier.

30. A medicament according to claim 29, which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

31. A medicament according to claim 30, in which (A) or (B), or (A) and (B), are in dispersion in the propellant, which is a halogen-substituted hydrocarbon.

32. A medicament according to claim 30, in which (A) or (B), or each of (A) and (B), has an average particle diameter of up to 10 μm.

33. A medicament according to claim 30, which is an inhalable nebulizable composition comprising a dispersion of (A) and (B) in an aqueous, organic or aqueous/organic medium or a combination of a dispersion of (A) in said medium with a dispersion of (B) in said medium.

34. A medicament according to claim 30, which is an inhalable dry powder comprising finely divided (A) or (B), or finely divided (A) and (B), optionally together with a pharmaceutically acceptable carrier in finely divided form.

35. A medicament according to claim 34, in which the carrier is present and is a saccharide.

36. A medicament according to claim 35, in which the carrier is lactose.

37. A medicament according to claim 36, in which (A) or (B), or each of (A) and (B), has an average particle diameter up to 10 microns.

38. The medicament according to claim 28, wherein the expectorant is one or more selected from the group consisting of Ambroxol, Guaiafenesin, Terpin hydrate, Potassium guaicolsulfonate and Carbocistiene.

39. A medicament containing, separately or together, (A) Pleconaril or a pharmaceutically acceptable salt thereof and (B) an NSAID, for simultaneous, sequential or separate administration in the treatment of an upper or lower respiratory, viral, inflammatory or obstructive airways disease.

40. A medicament according to claim 39 which is a pharmaceutical composition comprising a mixture of effective amounts of (A) and (B), optionally together with a pharmaceutically acceptable carrier.

41. A medicament according to claim 40, which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

42. A medicament according to claim 40, in which (A) or (B), or (A) and (B), are in dispersion in the propellant, which is a halogen-substituted hydrocarbon.

43. A medicament according to claim 40, in which (A) or (B), or each of (A) and (B), has an average particle diameter of up to 10 μm.

44. A medicament according to claim 40, which is an inhalable nebulizable composition comprising a dispersion of (A) and (B) in an aqueous, organic or aqueous/organic medium or a combination of a dispersion of (A) in said medium with a dispersion of (B) in said medium.

45. A medicament according to claim 40, which is an inhalable dry powder comprising finely divided (A) or (B), or finely divided (A) and (B), optionally together with a pharmaceutically acceptable carrier in finely divided form.

46. A medicament according to claim 45, in which the carrier is present and is a saccharide.

47. A medicament according to claim 46, in which the carrier is lactose.

48. A medicament according to claim 47, in which (A) or (B), or each of (A) and (B), has an average particle diameter up to 10 microns.

49. A pharmaceutical kit comprising (A) as defined in claim 39 and (B) as defined in claim 39 in separate unit dosage forms, said forms being suitable for administration of (A) and (B) in effective amounts, together with one or more inhalation devices for administration of (A) and (B).

50. The medicament according to claim 39, wherein the NSAID is one or more selected from the group consisting of Acetyl salicylic acid, Acetaminophen, Indomethacin, Diclofenac, Piroxicam, Tenoxicam, Ibuprofen, Naproxen, Ketoprofen, Nabumetone, Ketorolac, Azapropazone, Mefenamic acid, Tolfenamic acid, Sulindac, Diflunisal, Tiaprofenic acid, Podophyllotoxin derivatives, Acemetacin, Aceclofenac, Droxicam, Oxaprozin, Floctafenine, Phenylbutazone, Proglumetacin, Flurbiprofen, Tolmetin and Fenbufen.

51. A medicament containing, separately or together, (A) Pleconaril or a pharmaceutically acceptable salt thereof and (B) a decongestant, for simultaneous, sequential or separate administration in the treatment of an upper or lower respiratory, viral, inflammatory or obstructive airways disease.

52. A medicament according to claim 51 which is a pharmaceutical composition comprising a mixture of effective amounts of (A) and (B), optionally together with a pharmaceutically acceptable carrier.

53. A medicament according to claim 52 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

54. A medicament according to claim 53, in which (A) or (B), or (A) and (B), are in dispersion in the propellant, which is a halogen-substituted hydrocarbon.

55. A medicament according to claim 53, in which (A) or (B), or each of (A) and (B), has an average particle diameter of up to 10 μm.

56. A medicament according to claim 52, which is an inhalable nebulizable composition comprising a dispersion of (A) and (B) in an aqueous, organic or aqueous/organic medium or a combination of a dispersion of (A) in said medium with a dispersion of (B) in said medium.

57. A medicament according to claim 52, which is an inhalable dry powder comprising finely divided (A) or (B), or finely divided (A) and (B), optionally together with a pharmaceutically acceptable carrier in finely divided form.

58. A medicament according to claim 57, in which the carrier is present and is a saccharide.

59. A medicament according to claim 58, in which the carrier is lactose.

60. A medicament according to claim 51, in which (A) or (B), or each of (A) and (B), has an average particle diameter up to 10 microns.

61. A pharmaceutical kit comprising (A) as defined in claim 50 and (B) as defined in claim 50 in separate unit dosage forms, said forms being suitable for administration of (A) and (B) in effective amounts, together with one or more inhalation devices for administration of (A) and (B).

62. A medicament according to claim 50, wherein the decongestant is one or more selected from the group consisting of pseudoephedrine, phenypanolomine, levmetamfetamine, ephedrine, ephedrine hydrochloride, ephedrine sulfate, naphazoline hydrochloride, oxymetazoline hydrochloride, phenylephrine hydrochloride, propylhexedrine and xylometazoline hydrochloride.

63. A medicament containing, separately or together, (A) Pleconaril or a pharmaceutically acceptable salt thereof and (B) an anti-cholinergic agent, for simultaneous, sequential or separate administration in the treatment a respiratory, viral, inflammatory or obstructive airways disease.

64. A medicament according to claim 63 which is a pharmaceutical composition comprising a mixture of effective amounts of (A) and (B), optionally together with a pharmaceutically acceptable carrier.

65. A medicament according to claim 64 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

66. A medicament according to claim 65, in which (A) or (B), or (A) and (B), are in dispersion in the propellant, which is a halogen-substituted hydrocarbon.

67. A medicament according to claim 65, in which (A) or (B), or each of (A) and (B), has an average particle diameter of up to 10 μm.

68. A medicament according to claim 64, which is an inhalable nebulizable composition comprising a dispersion of (A) and (B) in an aqueous, organic or aqueous/organic medium or a combination of a dispersion of (A) in said medium with a dispersion of (B) in said medium.

69. A medicament according to claim 64, which is an inhalable dry powder comprising finely divided (A) or (B), or finely divided (A) and (B), optionally together with a pharmaceutically acceptable carrier in finely divided form.

70. A medicament according to claim 69, in which the carrier is present and is a saccharide.

71. A medicament according to claim 70, in which the carrier is lactose.

72. A medicament according to claim 64, in which (A) or (B), or each of (A) and (B), has an average particle diameter up to 10 microns.

73. A pharmaceutical kit comprising (A) as defined in claim 63 and (B) as defined in claim 63 in separate unit dosage forms, said forms being suitable for administration of (A) and (B) in effective amounts, together with one or more inhalation devices for administration of (A) and (B).

74. The medicament according to claim 63, wherein the anti-cholinergic compound is one or more selected from the group consisting of Tiotropium, Oxitropium, Ipratropium, Methantheline, Propantheline, Dicyclomine, Scopolamine, Methylscopolamine, Telenzepine, Benztropine, QNX-hemioxalate, Hexahydro-sila-difenidol hydrochloride and Pirenzepine.

75. A medicament containing, separately or together, (A) Pleconaril or a pharmaceutically acceptable salt thereof and (B) an P2Y₂ agonist, for simultaneous, sequential or separate administration in the treatment of an upper or lower respiratory, viral, inflammatory or obstructive airways disease.

76. A medicament according to claim 75 which is a pharmaceutical composition comprising a mixture of effective amounts of (A) and (B), optionally together with a pharmaceutically acceptable carrier.

77. A medicament according to claim 75 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

78. A medicament according to claim 77, in which (A) or (B), or (A) and (B), are in dispersion in the propellant, which is a halogen-substituted hydrocarbon.

79. A medicament according to claim 77, in which (A) or (B), or each of (A) and (B), has an average particle diameter of up to 10 μm.

80. A medicament according to claim 76, which is an inhalable nebulizable composition comprising a dispersion of (A) and (B) in an aqueous, organic or aqueous/organic medium or a combination of a dispersion of (A) in said medium with a dispersion of (B) in said medium.

81. A medicament according to claim 76, which is an inhalable dry powder comprising finely divided (A) or (B), or finely divided (A) and (B), optionally together with a pharmaceutically acceptable carrier in finely divided form.

82. A medicament according to claim 81, in which the carrier is present and is a saccharide.

83. A medicament according to claim 82, in which the carrier is lactose.

84. A pharmaceutical kit comprising (A) as defined in claim 75 and (B) as defined in claim 75 in separate unit dosage forms, said forms being suitable for administration of (A) and (B) in effective amounts, together with one or more inhalation devices for administration of (A) and (B).

85. A medicament containing, separately or together, (A) Pleconaril or a pharmaceutically acceptable salt thereof and (B) an antibiotic, for simultaneous, sequential or separate administration in the treatment of an upper or lower respiratory, viral, inflammatory or obstructive airways disease.

86. A medicament according to claim 85 which is a pharmaceutical composition comprising a mixture of effective amounts of (A) and (B), optionally together with a pharmaceutically acceptable carrier.

87. A medicament according to claim 86 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

88. A medicament according to claim 87, in which (A) or (B), or (A) and (B), are in dispersion in the propellant, which is a halogen-substituted hydrocarbon.

89. A medicament according to claim 87, in which (A) or (B), or each of (A) and (B), has an average particle diameter of up to 10 μm.

90. A medicament according to claim 86, which is an inhalable nebulizable composition comprising a dispersion of (A) and (B) in an aqueous, organic or aqueous/organic medium or a combination of a dispersion of (A) in said medium with a dispersion of (B) in said medium.

91. A medicament according to claim 86, which is an inhalable dry powder comprising finely divided (A) or (B), or finely divided (A) and (B), optionally together with a pharmaceutically acceptable carrier in finely divided form.

92. A medicament according to claim 91, in which the carrier is present and is a saccharide.

93. A medicament according to claim 92, in which the carrier is lactose.

94. A medicament according to claim 91, in which (A) or (B), or each of (A) and (B), has an average particle diameter up to 10 microns.

95. A pharmaceutical kit comprising (A) as defined in claim 85 and (B) as defined in claim 85 in separate unit dosage forms, said forms being suitable for administration of (A) and (B) in effective amounts, together with one or more inhalation devices for administration of (A) and (B).

96. A medicament according to claim 85, wherein the antibiotic is one or more selected from the group consisting of Tetracycline, Chlortetracycline, Bacitracin, Neomycin, Polymyxin, Gramicidin, Oxytetracycline, Chloramphenicol, Florfenicol, Gentamycin, Erythromycin, Clarithromycin, Azithromycin, Tulathromycin, or other suitable macrolide, Cefuroxime, Ceftibuten, Ceftiofur, Cefadroxil, or other suitable cephalosporin, Amoxicillin, Penicillins, Amoxicillin with clavulanic acid or an other suitable beta-lactamase inhibitor, antibacterials such as Sulfonamides, Sulfacetamide, Sulfamethizole, Sulfisoxazole; Nitrofurazone, and Sodium propionate.

97. A medicament containing, separately or together, (A) Pleconaril or a pharmaceutically acceptable salt thereof and (B) a Leukotriene₄ antagonist, for simultaneous, sequential or separate administration in the treatment of an upper or lower respiratory, viral, inflammatory or obstructive airways disease.

98. A medicament according to claim 97 which is a pharmaceutical composition comprising a mixture of effective amounts of (A) and (B), optionally together with a pharmaceutically acceptable carrier.

99. A medicament according to claim 98 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

100. A medicament according to claim 99, in which (A) or (B), or (A) and (B), are in dispersion in the propellant, which is a halogen-substituted hydrocarbon.

101. A medicament according to claim 99, in which (A) or (B), or each of (A) and (B), has an average particle diameter of up to 10 μm.

102. A medicament according to claim 98, which is an inhalable nebulizable composition comprising a dispersion of (A) and (B) in an aqueous, organic or aqueous/organic medium or a combination of a dispersion of (A) in said medium with a dispersion of (B) in said medium.

103. A medicament according to claim 98, which is an inhalable dry powder comprising finely divided (A) or (B), or finely divided (A) and (B), optionally together with a pharmaceutically acceptable carrier in finely divided form.

104. A medicament according to claim 103, in which the carrier is present and is a saccharide.

105. A medicament according to claim 104, in which the carrier is lactose.

106. A medicament according to claim 103, in which (A) or (B), or each of (A) and (B), has an average particle diameter up to 10 microns.

107. A pharmaceutical kit comprising (A) as defined in claim 97 and (B) as defined in claim 97 in separate unit dosage forms, said forms being suitable for administration of (A) and (B) in effective amounts, together with one or more inhalation devices for administration of (A) and (B).

108. A medicament containing, separately or together, (A) Pleconaril or a pharmaceutically acceptable salt thereof and (B) a compound selected from the group consisting of Zinc, echinaceia, Vitamin C and Vitamin E for simultaneous, sequential or separate administration in the treatment of an upper or lower respiratory, viral, inflammatory or obstructive airways disease.

109. A medicament according to claim 108 which is a pharmaceutical composition comprising a mixture of effective amounts of (A) and (B), optionally together with a pharmaceutically acceptable carrier.

110. A medicament according to claim 109 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

111. A medicament according to claim 110, in which (A) or (B), or (A) and (B), are in dispersion in the propellant, which is a halogen-substituted hydrocarbon.

112. A medicament according to claim 110, in which (A) or (B), or each of (A) and (B), has an average particle diameter of up to 10 μm.

113. A medicament according to claim 109, which is an inhalable nebulizable composition comprising a dispersion of (A) and (B) in an aqueous, organic or aqueous/organic medium or a combination of a dispersion of (A) in said medium with a dispersion of (B) in said medium.

114. A medicament according to claim 109, which is an inhalable dry powder comprising finely divided (A) or (B), or finely divided (A) and (B), optionally together with a pharmaceutically acceptable carrier in finely divided form.

115. A medicament according to claim 114, in which the carrier is present and is a saccharide.

116. A medicament according to claim 115, in which the carrier is lactose.

117. A medicament according to claim 114, in which (A) or (B), or each of (A) and (B), has an average particle diameter up to 10 microns.

118. A pharmaceutical kit comprising (A) as defined in claim 108 and (B) as defined in claim 108 in separate unit dosage forms, said forms being suitable for administration of (A) and (B) in effective amounts, together with one or more inhalation devices for administration of (A) and (B).

119. A medicament containing, separately or together, (A) Pleconaril or a pharmaceutically acceptable salt thereof and (B) a syk kinase antagonist, for sequential or separate administration in the treatment of a respiratory, viral, inflammatory or obstructive airways disease.

120. A medicament according to claim 119 which is a pharmaceutical composition comprising a mixture of effective amounts of (A) and (B), optionally together with a pharmaceutically acceptable carrier.

121. A medicament according to claim 120 which is an inhalable aerosol comprising a mixture of (A) and (B) in solution or dispersion in a propellant, or a combination of an aerosol containing (A) in solution or dispersion in a propellant with an aerosol containing (B) in solution or dispersion in a propellant.

122. A medicament according to claim 121, in which (A) or (B), or (A) and (B), are in dispersion in the propellant, which is a halogen-substituted hydrocarbon.

123. A medicament according to claim 121, in which (A) or (B), or each of (A) and (B), has an average particle diameter of up to 10 μm.

124. A medicament according to claim 120, which is an inhalable nebulizable composition comprising a dispersion of (A) and (B) in an aqueous, organic or aqueous/organic medium or a combination of a dispersion of (A) in said medium with a dispersion of (B) in said medium.

125. A medicament according to claim 120, which is an inhalable dry powder comprising finely divided (A) or (B), or finely divided (A) and (B), optionally together with a pharmaceutically acceptable carrier in finely divided form.

126. A medicament according to claim 125, in which the carrier is present and is a saccharide.

127. A medicament according to claim 126, in which the carrier is lactose.

128. A medicament according to claim 125, in which (A) or (B), or each of (A) and (B), has an average particle diameter up to 10 microns.

129. A pharmaceutical kit comprising (A) as defined in claim 119 and (B) as defined in claim 119 in separate unit dosage forms, said forms being suitable for administration of (A) and (B) in effective amounts, together with one or more inhalation devices for administration of (A) and (B).
